(19) United States
(12) Patent Application Publication
(10) Pub. No.: US 2022/0401068 A1
(43) Pub. Date: Dec. 22, 2022

(54) ULTRASOUND POLYMER GEL PAD AND METHOD OF USE

(71) Applicants: David John Treacey, Dubbo (AU); Rachell Jayne Haack, Dubbo (AU)

(72) Inventors: David John Treacey, Dubbo (AU); Rachell Jayne Haack, Dubbo (AU)

(21) Appl. No.: 17/353,382

(22) Filed: Jun. 21, 2021

(51) Int. Cl. A61B 8/00

(52) U.S. Cl. CPC A61B 8/4281

(57) ABSTRACT

A method for performing an ultrasound on a patient using an ultrasonic transducer has the steps of providing an ultrasound gel pad that includes a polymer gel body swollen in mineral oil, and positioning the ultrasound gel pad so that it, contacts both the patient and the ultrasonic transducer.

### BACKGROUND OF THE INVENTION

### FIELD OF THE INVENTION

This invention relates generally to ultrasound gel pads, and more particularly to an ultrasound gel pad that is swollen in mineral oil.

### DESCRIPTION OF RELATED ART

Ultrasound gel and gel pads are ordinarily constructed of polymer gels that are swollen with water. Aquaflax® aqueous gel pads, for example, are common gel pads that contain water and are used for ultrasound procedures.

Polymer gels containing mineral oil are also known in the art, but they are used for cushioning gels, such as are used in orthotics, insoles, prosthetics, etc. Eberle, U.S. Pat. No. 6,852,269 (assigned to Silipos, Inc.), teaches the use of such polymer materials, the disclosure of which is hereby incorporated by reference in full.

The prior art teaches the use of aqueous gel pads for ultrasound procedures. However, the prior art does not teach the use of polymer gel pads swollen in mineral oil for use in ultrasound procedures. The present invention fulfills these needs and provides further advantages as described in the following summary.

### SUMMARY OF THE INVENTION

The present invention teaches certain benefits in construction and use which give rise to the objectives described below.

The present invention provides a method for performing an ultrasound on a patient using an ultrasonic transducer. The method comprises the steps of providing an ultrasound gel pad that includes a polymer gel body swollen in mineral oil, and positioning the ultrasound gel pad so that it contacts both the patient and the ultrasonic transducer. The ultrasound imaging can then be performed, with improved imaging.

A primary objective of the present invention is to provide a ultrasound gel pad having advantages not taught by the prior art.

Another objective is to provide a method of using an ultrasound gel pad that contains mineral oil and is adapted for use with an ultrasonic transducer.

Another objective is to provide a method of using an ultrasound gel pad that provides superior imaging over other gels and gel pads.

Another objective is to provide a gel pad that is durable and resistant to tearing or other forms of wear and breakdown.

A further objective is to provide a method for performing an ultrasound on a patient using the improved gel pad.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention. In such drawings:

FIG. 1 is a perspective view of an ultrasound gel pad according to one embodiment of the present invention, illustrating use of the ultrasound gel pad on a patient;

FIG. 2 is a top plan view of the ultrasound gel pad;

FIG. 3 is a side elevational view thereof.

### DETAILED DESCRIPTION OF THE INVENTION

The above-described drawing figures illustrate the invention, an ultrasound gel pad and method of use.

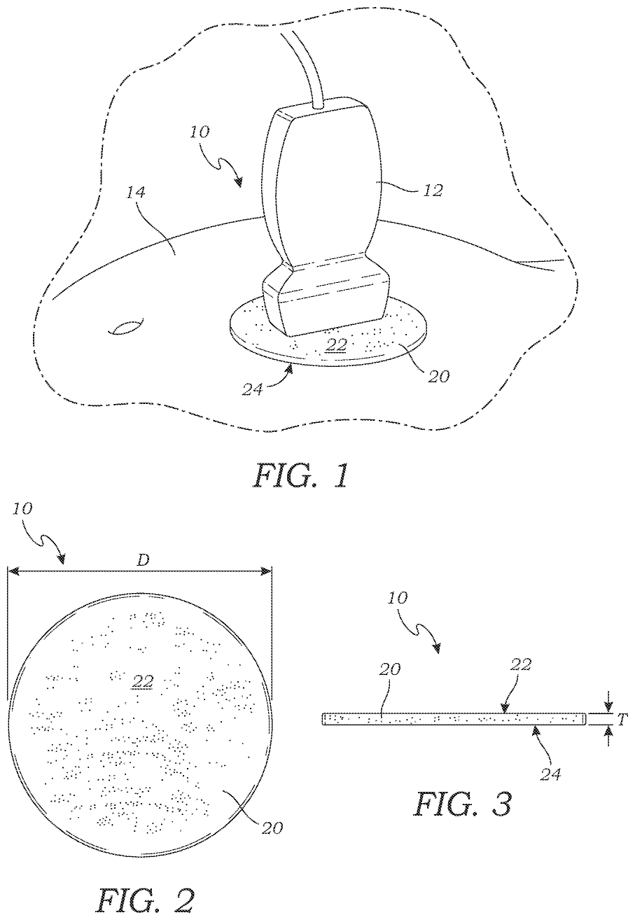

FIG. 1 is a perspective view of an ultrasound gel pad 10 according to one embodiment of the present invention, illustrating use of the ultrasound gel pad 10 on a patient 14. As shown in FIG. 1, the ultrasound gel pad 10 is used for a method for performing an ultrasound on the patient using an ultrasonic transducer 12. The ultrasound gel pad 10 functions as an alternative to the typical liquid gels positioned between the patient and the ultrasonic transducer 12 during ultrasonic transmission, and includes a polymer gel body 20 that is swollen in mineral oil.

The polymer gel body 20 is constructed of a polymeric material which may be in the form of a gel, a thermoplastic elastomer, or a combination thereof. Suitable thermoplastic elastomers include thermoplastic rubbers, and silicone containing elastomers. The polymer gel may be in the form of a block polymer. The material may include styrene-ethylenelbutylene-styrene block copolymers, or styrene-ethylene/propylene block copolymers. In another embodiment, styrene isoprenelbutadiene block copolymer is used. Other suitable polymers may be used, according to the discretion of those skilled in the art.

In another embodiment, the polymer gel may be a blend of a polystyrene-poly (ethylene-ethylene/propylene)-polystyrene block copolymer, sold under the trade name Polygel 51299 from PolyGel LLC, of Whippany, NJ.

The ratio of polymer to mineral oil can generally be between 1:1 to about 4:1, although this may vary depending upon the particular application. Further details of the ultrasound gel pad 10 are shown in FIGS. 2-3 and discussed below.

At a first step of a method of using the ultrasound gel pad 10, the polymer gel body 20 is positioned so that, it contacts both the patient 14 and the ultrasonic transducer 12. In this embodiment, the polymer gel body 20 includes top and bottom surfaces 22 and 24, wherein the top surface 22 abuts the ultrasonic transducer 12 during use, and the bottom surface 24 contacts the patient 14.

The ultrasonic transducer 12 transmits ultrasonic waves through the ultrasound gel pad 10 and into the patient 14. The ultrasound gel pad 10 provides superior imaging than prior art gel pads, and avoid air bubbles and other imperfections that may sometimes be caused by gels. The top and bottom surfaces 22 and 24 of the ultrasound gel pad 10 may be fully exposed and not covered with any obstructions, e.g., a fabric or paper layers, etc.

FIG. 2 is a top plan view of the ultrasound gel pad 10. In the embodiment shown in FIG. 2, the ultrasound gel pad 10 has a diameter D that is typically 7-15 cm, in this case about 9 cm. Alternatively, D may be much greater than 15 cm, for example, to cover a larger area of the patient's skin so that the gel pad 10 does not need to be moved to transmit ultrasonic waves to different areas. Or a large sheet may be cut to size prior to use. Alternative embodiments may be of any size, shape, and configuration that is suitable for the particular application.

While the figures illustrate a generally circular shape of the polymer gel body 20, the gel body 20 may be cut/formed in any desired shape, e.g., generally rectangular, any other shape or configuration to closely conform to a specific body part. For example, in the case of an ultrasound being performed on a person's leg, the gel body 20 may be adapted to conform around or on the leg (not shown). It would obviously be shaped differently if adapted for use on a hand, or other body part. The gel body 20 may be shaped to conform to any desired body part, as determined by one skilled in the art.

FIG. 3 is a side elevational view of the ultrasound gel pad 10. As shown in FIG. 3, the ultrasound gel pad 10 has a thickness T that is typically 0.3-3.0 cm, in this case 0.5-1.0 cm. Thickness T may be greater or lesser than this, depending on the construction of a sensor portion 16 of the ultrasonic transducer 12 (shown in FIG. 1), location of the ultrasonic transmission, and the desired results for optimal image quality.

While one embodiment of the ultrasound gel pad 10 is disclosed herein, this should not be construed to limit the scope of the invention to this single embodiment, but should be construed to encompass any alternative embodiments that would be obvious to one skilled in the art, given the teachings of the present invention. For example, the ultrasound gel pad 10 may be sold in a much larger sheet, and cut to any size or shape required by the user. The ultrasound gel pad 10 could also be sold in sizes and shapes suited for particular ultrasound procedures.

The ultrasound gel pad 10 could be secured on the transducer 12 with a probe cover. The ultrasound gel pad 10 could also be incorporated into a “sock” that fits over the ultrasound transducer 12, or other mounting mechanism for mounting the gel body 20 over the operative head (i.e., sensor portion 16) of the ultrasonic transducer 12, or alternatively to the person.

As used in this application, the words “a,” “an,” and “one” are defined to include one or more of the referenced item unless specifically stated otherwise. The terms “approximately” and “about” are defined to mean +/−10%, unless otherwise stated. Also, the terms “have,” “include,” “contain,” and similar tell is are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application. While the invention has been described with reference to at least one particular embodiment, it is to be clearly understood that the invention is not limited to these embodiments, but rather the scope of the invention is defined by claims made to the invention.

1. A method for performing an ultrasound on a patient using an ultrasonic transducer, the method comprising the steps of:
providing an ultrasound gel pad that includes a polymer gel body swollen in mineral oil, the polymer gel including styrene-ethylene/butylene-styrene block copolymers; and
positioning the ultrasound gel pad so that it contacts both the patient and the ultrasonic transducer.

2. The method of claim 1, further comprising the step of transmitting ultrasonic waves via the ultrasonic transducer through the ultrasound gel pad and into the patient.

3. The method of claim 1, wherein the ultrasound gel pad includes top and bottom surfaces, wherein the top surface abuts the ultrasonic transducer during use, and the bottom surface contacts the patient.

4. The method of claim 3, wherein the top and bottom surfaces of the ultrasound gel pad are fully exposed and not covered with any obstructions.

5. The method of claim 1, wherein the ultrasound gel pad has a diameter of 7-15 cm.

6. The method of claim 1, wherein the ultrasound gel pad has a thickness of 0.3-3.0 cm.

7. The method of claim 1, wherein the ultrasound gel pad has a thickness of 0.5-1.0 cm.
